(19) United States
(12) Patent Application Publication
(10) Pub. No.: US 2024/0409631 A1
(43) Pub. Date: Dec. 12, 2024

(54) AQUEOUS FORMULATIONS OF AN ANTI-CD22 ANTIBODY AND USES THEREOF

(71) Applicant: SINOMAB BIOSCIENCE LIMITED, Pak Shek Kok (NT), Hong Kong (CN)

(72) Inventors: Ming-Hon YAU, Hong Kong (CN); Kwan-Yin SIU, Hong Kong (CN); Ka-Wa Benny CHEUNG, Hong Kong (CN); Shui-on LEUNG, Hong Kong (CN)

(73) Assignee: SINOMAB BIOSCIENCE LIMITED, Pak Shek Kok (NT), Hong Kong (CN)

(21) Appl. No.: 18/701,828

(22) PCT Filed: Oct. 18, 2022
(86) PCT No.: PCT/IB2022/000593

§ 371 Date: Apr. 16, 2024

Related U.S. Application Data
(60) Provisional application No. 63/256,883, filed on Oct. 18, 2021.

(51) Int. Cl. C07K 16/28; A61K 39/00; A61K 47/26; A61P 37/06

(52) U.S. Cl. CPC C07K 16/2803; A61K 47/26; A61P 37/06; A61K 2039/505; A61K 2039/54; A61K 2039/545; C07K 2317/76

(57) ABSTRACT

A stable pharmaceutical formulation of a therapeutically active anti-CD22 antibody, Suciraslimab (or SM03), for injection. In particular, the formulations comprising, in addition to a suitable amount of the anti-CD22 antibody. The formulations comprise additionally at least one buffering agent, such as a phosphate buffer and a nonionic surfactant. Methods for preparing such stable formulation and their uses thereof are also provided.

### PRIORITY

The instant application corresponds to the U.S. National Phase of International Application No. PCT/IB2022/000593, filed, Oct. 18, 2022, which, in turn, claims the benefit of U.S. Provisional Application Ser. No. 63/256,883 filed Oct. 18, 2021, the entire contents of which are hereby incorporated by reference herein.

### SEQUENCE LISTING

The instant application contains a Sequence Listing that has been submitted as an ST.26 XML file via EFS-Web and is hereby incorporated by reference in its entirety. Said XML file, created on Oct. 18, 2022, is named_SBL-008PCT.xml and is 9,878 bytes in size.

### FIELD OF THE INVENTION

The present invention relates generally to aqueous formulations of an anti-CD-22 antibody, more particularly aqueous formulations of the SM03 antibody (Suciraslimab) suitable for injection or intravenous administration, and pharmaceutical uses thereof, particularly in connection with the treatment of a disease or disorder amenable to treatment with an anti-CD22 antibody.

### BACKGROUND OF THE INVENTION

Administration of anti-Siglec, specifically anti-CD22 antibodies, was shown to exhibit a therapeutic effect on various disorders, particularly autoimmune diseases such as rheumatoid arthritis (RA), rheumatoid spondylitis, systemic lupus erythematosus (SLE), multiple sclerosis (MS), autoimmune diabetes, Sjogren's syndrome or disease, psoriasis, inflammatory bowel disease, Graves' disease, diabetes mellitus type 1, celiac diseases, osteoarthritis and gouty arthritis, allergy, autoimmune uveitis, nephrotic syndrome, pemphigus vulgaris, Alzheimer's Disease, Behcet diseases (BD) and/or B cell malignancy. Administration of SM03, an anti-CD22 antibody referred to as “Suciraslimab”, to RA patients was found to improve disease associated complications by restoring the loss of regulatory and/or inhibitory functions of B cells, including different B cell subtypes such as regulatory B cells (Breg), due to insufficient conversion of CD22 and/or other Siglecs binding to their ligands from cis- to trans-configuration or unavailability of free ligand binding sites on human CD22 and/or other Siglecs due to these sites being “masked” by cis-ligand binding. However, there is an ongoing need for a stable pharmaceutical formulation of SM03 having an extended shelf life such that SM03 could be stored and transported with retention of its intended therapeutic activities. The present invention provides such a stable composition of SM03 suitable for therapeutic application. The pharmaceutical formulation of SM03 of the present invention is suitable for administrating to patient subjects via injection and/or infusion.

### SUMMARY OF THE INVENTION

There is a need in the art for a stable pharmaceutical formulations of an antibody which is suitable for therapeutic use by modulating CD22 and/or other Siglecs pathway that have an extended shelf life. There is further a need in the art for such a stable pharmaceutical formulation with an extended shelf life to be easily administered and to contain a high protein concentration.

Accordingly, it is an objective of the present invention is to provide such a liquid aqueous pharmaceutical formulation, more particularly an aqueous formulation that includes a therapeutically effective amount of an anti-CD22 antibody, specifically SM03 (or its humanized version, SM06), in a buffered solution forming a formulation having a pH between about 5.8 and about 8.0 and having a shelf life of at least 36 months. The present invention also includes an aqueous pharmaceutical formulation comprising a therapeutically effective amount of an antibody in a buffered solution forming a formulation having a pH between about 5.8 and 8.0 and having a shelf life of at least 18 months in the liquid state. In one embodiment, the pharmaceutical formulation has enhanced stability. In a further embodiment, the formulation of the present invention is stable following at least 3 freeze/thaw cycles of the formulation. In another embodiment, the antibody is directed to CD22. In yet another embodiment, the antibody of the present invention is directed to human CD22.

It is a further objective of the present invention to provide a liquid aqueous pharmaceutical formulation that includes a therapeutically effective amount of an antibody in a buffered solution forming a formulation having a pH between 5.8 and 8.0 and having enhanced stability of at least 12 months at a temperature of 2-8° C. In one embodiment, the formulation has enhanced stability of at least 18 months. In another embodiment, the antibody is directed to CD22. In yet another embodiment, the antibody is directed to human CD22.

In one embodiment of the present invention, the liquid aqueous pharmaceutical formulation is suitable for injection. In a further embodiment, the formulation is suitable for single use intravenous injection. In another embodiment, the concentration of the antibody in the liquid aqueous pharmaceutical formulation is about 5-25 mg/ml. In yet another embodiment, the concentration of the antibody in the formulation is about 10 mg/ml. In still another embodiment, the formulation has a high protein concentration. In yet another embodiment of the invention, the formulation is not light sensitive.

Another embodiment of the present invention includes a formulation where the antibody, or antigen-binding portion thereof, binds to human CD22.

In another embodiment of the present invention, the liquid aqueous pharmaceutical formulation contains an antibody, or a protein containing antigen-binding portion thereof, that is a recombinant antibody, or a protein containing antigen-binding portion thereof. In another embodiment, the formulation contains an antibody, or a protein containing antigen-binding portion thereof, that binds to human CD22 on human B-lymphocytes.

In yet another embodiment of the present invention, the liquid aqueous pharmaceutical formulation contains of an antibody, or a protein containing antigen-binding portion thereof, having a light chain variable region (LCVR) composed of a CDR3 domain comprising the amino acid sequence of SEQ ID NO: 3, or a modified version of SEQ ID NO: 3 that includes single alanine substitution at position 1, 4, 5, 7 or 8, along with a heavy chain variable region (HCVR) having a CDR3 domain comprising the amino acid sequence of SEQ ID NO: 4, or a modified version of SEQ ID NO: 4 that includes a single alanine substitution at position 2, 3, 4, 5, 6, 8, 9, 10 or 11. In a further embodiment, the formulation of the present invention contains an antibody, wherein the LCVR of the antibody, or an antigen-binding portion thereof, further has a CDR2 domain composed of the amino acid sequence of SEQ ID NO: 5, while the HCVR of the antibody, or an antigen-binding portion thereof, further has a CDR2 domain composed of the amino acid sequence of SEQ ID NO: 6. In yet another embodiment, the formulation of the present invention contains an antibody, wherein the LCVR of the antibody, or an antigen-binding portion thereof, further has CDR1 domain composed of the amino acid sequence of SEQ ID NO: 7 and the HCVR has a CDR1 domain composed of the amino acid sequence of SEQ ID NO: 8.

In yet another embodiment of the present invention, the antibody or antigen-binding portion thereof, contained in the liquid aqueous pharmaceutical formulation has a light chain variable region (LCVR) composed of the amino acid sequence of SEQ ID NO: 1 and a heavy chain variable region (HCVR) composed of the amino acid sequence of SEQ ID NO: 2. In another embodiment, the antibody, or antigen-binding portion thereof, has an IgG1 light chain constant region. In still another embodiment, the antibody, or antigen-binding portion thereof, has an IgG1 heavy chain constant region. In another embodiment, the antibody, or antigen-binding portion thereof, is a Fab fragment. In still a further embodiment, the antibody, or antigen-binding portion thereof, is a single chain Fv fragment.

In one embodiment of the present invention, the liquid aqueous pharmaceutical formulation contains an antibody, or a protein containing antigen-binding portion thereof, that binds to human CD22 and at least one additional primate CD22 selected from the group consisting of baboon CD22, marmoset CD22, cynomolgus CD22, and rhesus CD22.

It is yet a further objective of the present invention to provide an aqueous pharmaceutical composition comprising a surfactant and a buffer system that contains phosphate with a pH of about 5.8 to 8.0, in amounts sufficient to formulate an antibody for therapeutic use at a concentration of greater than about 5 mg/ml. In one embodiment, the surfactant is polysorbate 80. In another embodiment, the composition includes 0.01 to 0.08% of polysorbate 80. In a further embodiment, the composition includes the antibody SM03.

It is yet a further objective of the present invention to provide a liquid aqueous pharmaceutical formulation composed of 0.01 to 0.08% of Tween-80, and a buffer system that contains phosphate, with a pH of 5.8 to 8.0. In one embodiment, the antibody is directed to CD22. In another embodiment, the formulation contains about 10 mg/ml of antibody. The present invention further provides a liquid aqueous pharmaceutical formulation composed of about 10 mg/ml of antibody about 0.04% of Tween-80, and a buffer system containing phosphate, with a pH of about 5.8 to about 8.0. In one embodiment, the pH of the formulation ranges between about 6.2 to about 7.7. In another embodiment, the pH falls between about 6.5 to about 7.5. In yet another embodiment, the pH of the invention falls between about 6.9 to about 7.2.

In one embodiment of the present invention, the liquid aqueous pharmaceutical formulation also includes about 7.16 mg/ml of disodium phosphate dodecahydrate, about 1.38 mg/ml of sodium dihydrogen phosphate. In another embodiment, the formulation of the present invention includes an antibody that is directed to CD22. In yet a further embodiment, the formulation of the present invention is administered to a subject suffering from autoimmune disorders such as rheumatoid arthritis, systemic lupus erythematosus, non-Hodgkin's lymphoma, etc.

These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims.

### BRIEF DESCRIPTION OF THE DRAWINGS

FIG. 1 graphically depicts the results of the trials of Example 4 wherein an intravenous formulation of SM03 was administered biweekly to patients with rheumatoid arthritis.

### DETAILED DESCRIPTION OF THE PREFERRED EMBODIMENTS

The present invention pertains to a liquid aqueous pharmaceutical formulation having a pH of about 5.8 to about 8.0 that contains a high protein concentration, including an antibody concentration ranging from about 1 to about 25 mg/ml, and has enhanced stability. The present invention also pertains to a liquid aqueous pharmaceutical formulation for the treatment of subject suffering from an auto-immune disorder. The formulation of the present invention preferably includes the following constituents: an antibody that binds to human CD22 with a high affinity, low off rate and high internalization stimulating rate; a buffer, which includes disodium phosphate dodecahydrate, and sodium dihydrogen phosphate; a surfactant, including polysorbate 80 or polysorbate 20; and sodium hydroxide or hydrochloric acid, for pH adjustment.

### I. Definitions

Unless otherwise defined, all technical and scientific terms used herein have the same meaning as commonly understood by one of ordinary skill in the art to which this invention belongs. However, in case of conflict, the present specification, including definitions, will control. Accordingly, in the context of the present invention, the following definitions apply:

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a human.

The terms “pharmaceutical formulation” and “pharmaceutical composition” are used interchangeably herein to refer to preparations that are in such form as to permit the biological activity of the active ingredients to be unequivocally effective, and which contain no additional components which are significantly toxic to the subjects to which the formulation would be administered. Likewise, the term “pharmaceutically acceptable” excipients (vehicles, additives) refers to those that can reasonably be administered to a subject to provide an effective dose of the active ingredient employed.

A “stable” formulation is one in which the antibody therein essentially retains its physical stability and/or chemical stability and/or biological activity upon storage. Various analytical techniques for measuring stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Preferably, the formulation of the present invention is stable at room temperature (about 15-25° C.) for at least 6 month, and/or stable at about 2-8° C. for at least 3 years (36 months). Furthermore, the formulation of the present invention is preferably stable following freezing (to, e.g., lower than −40° C.) and thawing of the formulation, hereinafter referred to as a “freeze/thaw cycle (F/T cycle)”.

An antibody “retains its physical stability” in a pharmaceutical formulation if it shows substantially no signs of aggregation, precipitation and/or denaturation upon visual examination of color and/or clarity, or as measured by UV light scattering or by size exclusion chromatography.

An antibody “retains its chemical stability” in a pharmaceutical formulation, if the chemical stability at a given time is such that the antibody is considered to still retain its biological activity as defined below. Chemical stability can be assessed by detecting and quantifying chemically altered forms of the antibody. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography, for example.

An antibody “retains its biological activity” in a pharmaceutical formulation if the antibody in a pharmaceutical formulation is biologically active for its intended purpose. For example, biological activity is retained if the biological activity of the antibody in the pharmaceutical formulation is within about 30%, about 20%, or about 10% (within the errors of the assay) of the biological activity exhibited at the time the pharmaceutical formulation was prepared (e.g., as determined in an antigen binding assay).

As used herein, “buffer” refers to a buffered solution that resists changes in pH by the action of its acid-base conjugate components. The buffer of this invention has a pH in the range from about 5.8 to about 8.0; preferably from about 6.5 to about 7.5; and most preferably has a pH in the range from about 6.9 to about 7.2. Examples of buffers that will control the pH in this range include acetate (e.g. sodium acetate), succinate (such as sodium succinate), phosphate (such as sodium phosphate), histidine, citrate and other organic salt buffers.

As used herein and in the claims, the term “comprising” means including the following elements but not excluding others.

The term “auto-immune disease or disorder” refers to a disease or disorder characterized by an abnormal immune response to normal body cells, tissue, organ, or a whole body part. For instance, the immune system may produce antibodies that attack normal body tissue. In some embodiments, the auto-immune disease or disorder is caused by dysfunctional immune control. An auto-immune disease or disorder includes, but is not limited to, rheumatoid arthritis (RA), systemic lupus erythematosus (SLE), multiple sclerosis (MS), Sjogren's syndrome, psoriasis, inflammatory bowel disease, Graves' disease, diabetes mellitus type 1, celiac diseases, rheumatoid spondylitis, osteoarthritis and gouty arthritis, allergy, autoimmune uveitis, nephrotic syndrome, pemphigus vulgaris, Alzheimer's Disease, Behcet diseases (BD), and B cell malignancy, etc.

The term “CD22” refers to a B cell restricted antigen that belongs to the immunoglobulin (Ig) superfamily and is a type I transmembrane sialoglycoprotein also known as sialic-acid-binding immunoglobulin-type lectins (Siglecs) with a molecular size of 135 kD. In resting B cells, CD22 is a prominent cis ligand for itself, forming CD22 homo-oligomers.

The term “Siglecs”, as used herein, refers to a sialic-acid-binding immunoglobulin-type lectins such as human CD22 and Siglec-10. Siglec-10 and CD22 are the only Siglecs expressed on B-cell surfaces. These molecules are constitutively expressed on B cells and act as inhibitory co-receptors of the B cell antigen receptor (BCR) in the maintenance of B-cell tolerance and prevention of autoimmune diseases.

The term “human CD22” or “hCD22” as used herein, is intended to refer to a human B cell-restricted surface antigen CD22 that exists as a type I transmembrane sialoglycoprotein with a molecular size of 135 kD. CD22 is another B cell restricted antigen; it belongs to the immunoglobulin (Ig) superfamily and is a type I transmembrane sialoglycoprotein with a molecular size of 135 kD. The term human CD22 is intended to include recombinant human CD22 (rhCD22), either in its full molecular form containing the extracellular, intracellular and transmembrane regions, or expressed as a soluble form containing only the extracellular regions, or portions of the extracellular portions containing the binding epitopes of the anti-CD22 antibodies, which can be prepared by standard recombinant expression methods or purchased commercially (e.g., Abcam, Catalog No. Ab50033, Cambridge, MA; Creative Biomart, Catalog No. CD22-3946H, Shirely, NY; Thermo Fisher Scientific, Catalog No. 11958H08H5, Waltham, MA)

In a pharmacological sense, in the context of the present invention, the phrases “therapeutically effective amount” and “effective amount”, particularly of an antibody, are used interchangeably herein to refer to an amount effective in the prevention or treatment of a disorder for the treatment of which the antibody is effective. A “disorder” is any condition that would benefit from treatment with the antibody. This includes chronic and acute disorders or diseases including those pathological conditions which predisposes the subject to the disorder in question.

“Treatment” refers to both therapeutic treatment and prophylactic or preventative measures. Those in need of treatment include those already with the disorder as well as those in which the disorder is to be prevented.

The term “antibody”, as used herein, is intended to refer to immunoglobulin molecules comprised of four polypeptide chains, two heavy (H) chains and two light (L) chains interconnected by disulfide bonds. Each heavy chain is comprised of a heavy chain variable region (abbreviated herein as HCVR or VH) and a heavy chain constant region. The heavy chain constant region is comprised of three domains, CH1, CH2 and CH3, and there is a short flexible hinge region connecting the CH1 and CH2 domains. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “recombinant antibody”, as used herein, is intended to include all chimeric and humanized antibodies that are prepared, expressed, or created or isolated by recombinant means, such as antibodies expressed using a recombinant expression vector transfected into a host cell (described further in Section II, below), antibodies isolated from a recombinant, combinatorial human antibody library (described further in Section III, below), antibodies isolated from an animal (e.g., a mouse) that is transgenic for human immunoglobulin genes (see e.g., Taylor, L.D., et al (1992) Nucl. Acids Res . 20: 6287-6295) or antibodies prepared, expressed, created or isolated by any other means that involves splicing of human immunoglobulin gene sequences to other DNA sequences.

An “isolated antibody”, as used herein, is intended to refer to an antibody that is substantially free of other antibodies having different antigenic specificities (e.g., an isolated antibody that specifically binds human CD22 is substantially free of antibodies that specifically bind antigens other than human CD22). An isolated antibody that specifically binds human CD22 may, however, have cross-reactivity to other antigens, such as CD22 from other species. Moreover, an isolated antibody may be substantially free of other cellular material and/or chemicals.

The term “SM03” refers to a chimeric antibody against human CD22 (hCD22). Such an antibody is described in Leung et al. in Chinese Pat. No. ZL03123054.7 and U.S. Pat. Nos. 9,371,396 B2 and 10,613,099 B2, the contents of each of which are incorporated by reference herein their entirety.

The term “SM06” refers to a humanized version of chimeric antibody SM03 reengineered to reduce its potential immunogenicity and exhibiting affinity and specificity against human CD22 comparable to that of SM03 (See Liang et al. (2006) Chinese J New Drug 15 (21): 1832-1836). The construction of the framework-patched SM06 and its uses are described in Chinese Patent No. ZL 011 44894.6 as well as US Pat. Nos. 7,321,026 B2and 7,338,659 B2, the contents of each of which are incorporated by reference herein their entirety.

The term “isolated antibody”, as used herein, is intended to refer to an antibody that is substantially free of other antibodies having different antigenic specificities (e.g., an isolated antibody that specifically binds CD22 is substantially free of antibodies that specifically bind antigens other than CD22). An isolated antibody that specifically binds CD22 may, however, have cross-reactivity to other antigens, such as CD22 molecules from other species. Moreover, an isolated antibody may be substantially free of other cellular material and/or chemicals.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The terms “polynucleotides”, “oligonucleotides” “nucleotides”, “nucleic acids”, and “nucleic acid molecules” are used interchangeably herein to refer to a polymer of nucleic acid residues linked by ester bonding, and, unless otherwise specifically indicated, are similarly to the amino acids referred to by their commonly accepted single-letter codes. Similar to the amino acids, they encompass both naturally-occurring and non-naturally occurring nucleic acid polymers. The polynucleotide or oligonucleotide may be composed of DNA, RNA or a combination thereof.

In the context of the present invention, nucleic acid sequences are deemed “substantially identical” where they have between about 70% and about 80% or more preferably, between about 81% and about 90%, or even more preferably, between about 91% and about 99%, sequence identity for a particular oligonucleotides.

The term “isolated” is used herein to refer to a molecule separated from its native environment (i.e., in a non-naturally occurring form). Likewise, the term “purified” is used herein to refer to a molecule in a form substantially free from contaminants.

### II. Antibodies of a Formulation of the Present Invention

The present invention is directed to a liquid aqueous pharmaceutical formulation containing a therapeutically effective amount of an antibody in a buffered solution so as to yield a formulation having a pH between about 5.8 and about 8.0 and an extended shelf life, preferably of at least about 18 months. In another embodiment, the liquid aqueous pharmaceutical formulation of the invention has enhanced stability. In a further embodiment of the present invention, the formulation is not light sensitive. In yet another embodiment of the present invention, the claimed formulation remains stable following at least three F/T cycles. In still another embodiment, the pharmaceutical formulation of the invention is suitable for single use intravenous (iv) injection.

Antibodies that can be used in the formulation of the present invention include polyclonal, monoclonal, recombinant antibodies, single chain antibodies, hybrid antibodies, chimeric antibodies, humanized antibodies, or fragments thereof. Antibody-like molecules containing one or two binding sites for an antigen and a Fc-part of an immunoglobulin can also be used. Preferred antibodies used in the formulation are recombinant chimeric or humanized antibodies, especially preferred antibodies directed against the antigen CD22, including human CD22 (hCD22). In another embodiment, the recombinant antibody used in the formulation is preferred to be a preparation of isolated antibody directed against the antigen CD22.

In another embodiment, the recombinant antibody is SM03 (chimeric) or SM06 (humanized version), and has a light chain CDR3 domain comprised of the amino acid sequence of SEQ ID NO: 4 and a heavy chain CDR3 domain comprised of the amino acid sequence of SEQ ID NO: 8 (set forth in the attached Sequence Listing). In some embodiments, the SM03 antibody has a light chain variable region (LCVR) comprised of the amino acid sequence of SEQ ID NO: 1 and a heavy chain variable region (HCVR) comprised of the amino acid sequence of SEQ ID NO: 5; whereas the SM06 antibody has a light chain variable region (LCVR) comprised of the amino acid sequence of SEQ ID NO: 9 and a heavy chain variable region (HCVR) comprised of the amino acid sequence of SEQ ID NO: 010. These antibodies are described in Chinese Pat. Nos. ZL 031 23054.7 and ZL 011 44894.6 and US Pat. Nos. 7,321,026 B2, 7,338,659 B2, 9,371,396 B2 and 10,613,099 B2, the contents of each of which are incorporated by reference herein in their entirety.

### III. Preparation of a Formulation of the Present Invention

The present invention features formulations (e.g., protein formulations and/or antibody formulations) having improved properties as compared to art-recognized formulations. For example, the formulations of the invention have an improved shelf life and/or stability as compared to art recognized formulations. In a preferred aspect, the formulations of the invention have a high protein concentration, including, for example, a protein concentration greater than about 25 mg/ml, a protein concentration greater than about 30 mg/ml, a protein concentration greater than about 50 mg/ml, or a protein concentration greater than about 100 mg/ml. In a preferred embodiment of the invention, the protein is an antibody. In another preferred embodiment, the antibody is SM03. The present invention also provides an aqueous pharmaceutical composition that includes a surfactant and a buffer system comprising phosphate with a pH of about 5.8 to 8.0, in amounts sufficient to formulate an antibody for therapeutic use at a concentration of greater than about, for example, 5 mg/ml.

Preparation of the antibody of interest is performed according to standard methods known in the art. In a preferred embodiment of the present invention, the antibody used in the formulation is expressed in CHO or Sp2/0 cells and purified by a standard series of chromatography steps. In a further preferred embodiment, the antibody is directed to mammalian CD22.

After preparation of the antibody of interest, preferred to be a preparation of isolated antibody, the pharmaceutical formulation comprising the antibody is prepared. The therapeutically effective amount of antibody present in the formulation is determined, for example, by taking into account the desired dose volumes and mode(s) of administration. In one embodiment of the invention, the concentration of the antibody in the formulation is between about 1 to about 100 mg of antibody per ml of liquid formulation. In a preferred embodiment, the concentration of the antibody in the formulation is between about 5 to about 50 mg per ml. In another preferred embodiment, the concentration of the antibody in the formulation is between about 10 to about 25 mg/ml. The formulation is especially suitable for large antibody dosages of more than 8 mg/ml. In a preferred embodiment, the concentration of the antibody is 10 mg/ml.

In another embodiment of the invention, the concentration of the antibody in the formulation is about 1-100 mg/ml, about 5-95 mg/ml, about 5-90 mg/ml, about 5-85 mg/ml, about 5-80 mg/ml, about 5-75 mg/ml, about 5-70 mg/ml, about 5-65 mg/ml, about 5-60 mg/ml, about 5-55 mg/ml, about 5-50 mg/ml, about 5-45 mg/ml, about 5-40 mg/ml, about 5-35 mg/ml, about 5-30 mg/ml, about 5-25 mg/ml, about 8-15 mg/ml, or about 10 mg/ml. Ranges intermediate to the above recited concentrations, e.g., about 8-94 mg/ml, are also intended to be part of this invention. For example, ranges of values using a combination of any of the above recited values as upper and/or lower limits are intended to be included.

In one embodiment, the present invention provides a formulation with an extended shelf life that contains of an active ingredient, preferably an antibody, in combination with disodium phosphate dodecahydrate, sodium dihydrogen phosphate, polysorbate 80, water for injection, hydrochloric acid and sodium hydroxide. In a further embodiment, the formulation of the invention has an extended shelf life of at least about 18 months in the liquid state. Freezing the formulation of the invention can also be used to further extend its shelf life.

An aqueous formulation of the present invention may include the antibody in a pH-buffered solution. The buffer of the present invention has a pH ranging from about 5.8 to about 8.0, preferably from about 6.5 to about 7.5, more preferably from about 6.9 to about 7.2, and most preferably has a pH of about 7.0 to about 7.2. Ranges intermediate to the above recited pH values are also intended to be part of this invention. For example, ranges of values using a combination of any of the above recited values as upper and/or lower limits are intended to be included. Examples of buffers that will control the pH within this range include phosphate (e.g. sodium phosphate), acetate (e.g. sodium acetate), succinate (such as sodium succinate), gluconate, histidine, citrate and other organic salt buffers.

In a preferred embodiment of the invention, the formulation comprises a buffer system that contains citrate and phosphate to maintain the pH in a range of about 5.8 to about 8.0, preferably from about 6.5 to about 7.5, more preferably from about 6.9 to about 7.2, and most preferably has a pH of about 7.0 to about 7.2. In another preferred embodiment, the buffer system includes disodium phosphate dodecahydrate, and sodium dihydrogen phosphate. In a further preferred embodiment, the buffer system includes about 1.2 mg/ml of sodium dihydrogen phosphate (e.g. 1.0 to 1.4 mg/ml), about 3.6 mg/ml of disodium phosphate dihydrate (e.g. 3.4-3.8 mg/ml). In additional preferred embodiments, the buffer system includes 1.1 to 1.5 mg/ml of sodium dihydrogen phosphate monohydrate, or 1.3 to 1.7 mg/ml of sodium dihydrogen phosphate dihydrate and 7.0 to 7.4 mg/ml of disodium phosphate dodecahydrate. In another preferred embodiment, about 10 mmol/L (10 mM) of sodium dihydrogen phosphate and 20 mmol/L (20 mM) of disodium phosphate are found in the formulation of the invention. In a further embodiment, the pH of the formulation is adjusted with sodium hydroxide solution and/or hydrochloric acid.

A detergent or surfactant may be added to the antibody formulation. Exemplary detergents include nonionic surfactant such as polysorbates (e.g. polysorbates 20, polysorbate 80, Tween 20, Tween 80 etc). The amount of surfactant added is such that it reduces aggregation of the formulated antibody and/or minimizes the formation of particulates in the formulation and/or reduces adsorption. In a preferred embodiment of the invention, the formulation includes a surfactant which is a polysorbate. In another preferred embodiment of the invention, the formulation contains the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In one preferred embodiment, the formulation contains between about 0.1 and about 0.8mg/ml of polysorbate 80, more preferably between about 0.3 and about 0.5 mg/ml. In another preferred embodiment, about 0.4% polysorbate 80 is found in the formulation of the invention.

In a preferred embodiment of the present invention, the formulation is enclosed in a vial in form of solution at about 30 ml containing the ingredients per vial as shown below in Table 1.

TABLE 1
1 vial with 30 ml solution for injection contains:

| Name of ingredient | Quantity | Unit | Function |
|---|---|---|---|
| Antibody | 300 | mg | Active substance |
| Disodium phosphate | 214.8 | mg | Excipient; pH |
| dodecahydrate | | | buffering |
| Sodium dihydrogen | 41.4 | mg | Excipient; pH |
| phosphate monohydrate | | | buffering |
| Polysorbate 80 | 12 | mg | Surfactant |
| Water for injections | 30000 | mg | Solvent |
| Sodium hydroxide²⁾ | 3-15 | mg | pH adjustment |
| Hydrochloric acid²⁾ | 3-15 | mg | pH adjustment |

¹⁾Density of the solution: 1.020 g/mL
²⁾Addition as 1M solution

The formulation herein may also be combined with one or more other therapeutic agents or pharmaceutically acceptable carriers as necessary for the particular indication being treated, preferably those with complementary activities that do not adversely affect the antibody of the formulation. Such therapeutic agents or pharmaceutically acceptable carriers are suitably present in combination in amounts that are effective for the purpose intended.

The formulations to be used for in vivo administration must be sterile. This is readily accomplished by filtration through sterile filtration membranes, prior to, or following, preparation of the formulation.

### IV. Administration of a Formulation of the Present Invention

Herein, the phrase “effective amount” in reference to the formulation of the present invention means that an amount effective in the prevention or treatment of a disorder, e.g., to prevent the various morphological and somatic symptoms of an auto-immune disorder, is present in the formulation. In another embodiment, the “effective amount” of the formulation refers to the amount necessary or sufficient to modulate B-lymphocyte activity, e.g., prevent the various morphological and somatic symptoms of a detrimental B lymphocyte activity-associated state. In another embodiment, the effective amount of the formulation refers to the amount necessary to achieve the desired result.

In one example, an effective amount of the formulation is the amount sufficient to modulate detrimental CD22 activity. In another example, an effective amount of the formulation is the amount sufficient to modulate detrimental B lymphocyte activity. In yet another example, an effective amount of the formulation is 30 ml of the formulation containing 300 mg of antibody, as described in Table 1. The effective amount can vary depending on such factors as the size and weight of the subject, or the type of illness. In another example, an effective amount of the formulation is 60 ml of the formulation containing 600 mg of antibody.

The regimen of administration can affect what constitutes an effective amount. The anti-CD22 antibody formulation can be administered to the subject either prior to or after the onset of detrimental B lymphocyte activity.

The term “treated,” “treating” or “treatment” includes the diminishment or alleviation of at least one symptom associated or caused by the state, disorder or disease being treated. For example, treatment can be diminishment of one or several symptoms of a disorder or complete eradication of a disorder.

Actual dosage levels of the active ingredient(s) (i.e., at least one antibody) in the pharmaceutical formulations of the present invention may be varied so as to obtain an amount of the active ingredient that is effective to achieve the desired therapeutic response for a particular patient, composition, and mode of administration, without being toxic to the patient. The selected dosage level will depend upon a variety of factors including the activity of the antibody found in the formulation, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition of the present invention required. For example, the physician or veterinarian could start doses of the compounds of the invention employed in the pharmaceutical formulation at levels lower than that required in order to achieve the desired therapeutic effect and gradually increase the dosage until the desired effect is achieved.

In general, a suitable daily dose of a formulation of the present invention will be that amount of the formulation that is the lowest dose effective to produce a therapeutic effect. Such an effective dose will generally depend upon the factors described above. An effective amount of the formulation of the present invention is an amount that modulates CD22 activity in a subject suffering from auto-immune disorder. In a preferred embodiment, the formulation provides an effective dose of 300 mg per infusion of the active ingredient, the antibody. In another embodiment, the formulation provides an effective dose which ranges from about 1 to 600 mg of antibody.

It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The present invention provides a pharmaceutical formulation with an extended shelf life, which, in one embodiment, is used to modulate CD22 activity in a subject suffering from a disorder in which B lymphocyte activity is detrimental, comprising administering to the subject an antibody or antibody portion of the invention such that CD22 activity in the subject is modulated. Preferably, the CD22 is hCD22 and the subject is a human subject. Alternatively, the subject can be a mammal expressing a CD22 with which an antibody of the invention cross-reacts. Still further the subject can be a mammal into which has been introduced hCD22 (e.g., by administration of hCD22 or by expression of an hCD22 transgene). A formulation of the present invention can be administered to a human subject for therapeutic purposes (discussed further below). In a preferred embodiment, the formulation of the present invention is administered through intravenous infusion, preferably single use. Moreover, a formulation of the present invention can be administered to a non-human mammal expressing a CD22 with which the antibody cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration).

As used herein, the term “a disorder in which CD22 activity is detrimental” is intended to include diseases and other disorders in which the presence of CD22 in a subject suffering from the disorder has been shown to be or is suspected of being either responsible for the pathophysiology of the disorder or a factor that contributes to a worsening of the disorder. Accordingly, a disorder in which CD22 activity is detrimental is a disorder in which inhibition of CD22 activity is expected to alleviate the symptoms and/or progression of the disorder. Such disorders may be evidenced, for example, by an increase in the concentration of CD22 in a biological fluid of a subject suffering from the disorder (e.g., an increase in the concentration of CD22 in serum, plasma, synovial fluid, etc. of the subject), which can be detected, for example, using an anti-CD22 antibody as described above.

The use of a pharmaceutical formulation containing anti-CD22 antibodies of the invention in the treatment of specific autoimmune disorders is discussed further below.

### V. Therapeutic Uses of a Formulation of the Present Invention

The formulation of the present invention can be used to treat autoimmune diseases, in particular those associated with disrupted immune self-tolerance, including rheumatoid arthritis, rheumatoid spondylitis, osteoarthritis and gouty arthritis, allergy, multiple sclerosis, autoimmune diabetes, autoimmune uveitis and nephrotic syndrome. Typically, the inventive formulation is administered systemically, alone or in combination.

The pharmaceutical formulation of the present invention, also can be used to treat various other disorders in which CD22 activity is detrimental. Examples of other diseases and disorders in which CD22 activity has been implicated in the pathophysiology, and thus which can be treated using the formulation of the invention.

Hereinafter, the present invention is described in more detail by reference to the Examples. However, the following materials, methods and examples only illustrate aspects of the invention and in no way are intended to limit the scope of the present invention. As such, methods and materials similar or equivalent to those described herein can be used in the practice or testing of the present invention.

### EXAMPLES

The aqueous formulation of anti-CD22 antibody of the present invention was developed based on the experimental results as provided below using the general preparatory and analytical methods and assays as outlined below.

### A. Preparation of the Antibody for the Formulation

SM03 is produced by general techniques known for the production of recombinant proteins. A genetically engineered animal cell line, preferably Chinese hamster ovary (CHO) or Sp2/0 cell line, was prepared and expanded in cell culture from a master cell bank or working cell bank. The SM03 monoclonal antibody is harvested from the cell culture fluid and purified using affinity chromatography preferably Protein A affinity chromatography, followed by a low pH viral inactivation (e.g. pH lower than 4.0), an anion exchange chromatography (e.g. Q-Sepharose FF), a cation exchange chromatography (e.g. SP-Sepharose FF), a filtration step to remove viral contaminations (e.g. a hollow fiber), and an ultrafiltration/diafiltration step. SM03 was then prepared into a concentration of approx. 10 mg/ml in a 30 mM phosphate buffer at a pH of approximately 7.2.

The process begins with thawing of cells from the working cell bank (WCB) or master cell bank (MCB) and expansion through cell culture in a series of shake flasks and spinner flasks followed by expansion in a series of bioreactors with increasing culture volume. After completion of the bioreactor production phase, the cell culture liquid is clarified by centrifugation and/or filtration. The protein is then purified by a series of column chromatography processes to remove process and product related impurities. A viral filtration step is performed, and the filtered bulk is then concentrated, formulated by ultrafiltration/diafiltration, preferably by tangential flow filtration, into the final buffer: 10˜100 mg/ml SM03 in 30 mM phosphate buffer, pH 7.2, 0.04% (w/v) polysorbate 80. The resultant drug substance bulk is stored at 2˜8° C. and stable for not less than 6 months.

The other excipients of the formulation in accordance with the present invention are widely used in the practice and known to the person skilled in the art. There is therefore no need for them to be explained here in detail.

Liquid drug product formulations for subcutaneous administration according to the invention were developed as follows.

### Example 1—Preparation of the Formulation

The aqueous pharmaceutical formulation of the invention described in Table 1 was made according to the following process:

Materials which were used in the formulation include: disodium phosphate dodecahydrate, sodium dihydrogen phosphate monohydrate, polysorbate 80 and water for injection (WFI). Sodium hydroxide and hydrochloric acid which were used as a 1M solution for pH adjustment, and Suciraslimab (SM03) drug substance bulk.

Preparation of 20 L of Diafiltration Buffer (Equivalent to 20.180 kg—Density of the Solution: 1.009 g/ml): Ingredients were weighed out as follows: 143.2 g disodium phosphate dodecahydrate, 17.2 g sodium dihydrogen phosphate, and 19,959.6 g of water.

Preparation of 20 L of Formulation Adjusting Buffer (Equivalent to 20.180kg—Density of the Solution: 1.009 g/ml): Ingredients were weighed out as follows: 143.2 g disodium phosphate dodecahydrate, 17.2 g sodium dihydrogen phosphate, 40.0 g polysorbate 80, and 19,919.6 g of water.

Preparation of 20 L of Formulation Buffer (Equivalent to 20.180 kg—Density of the Solution: 1.009 g/ml): Ingredients were weighed out as follows: 143.2 g disodium phosphate dodecahydrate, 17.2 g sodium dihydrogen phosphate, 8.0 g polysorbate 80, and 19,959.6 g of water.

A 1M sodium hydroxide solution was prepared by combining 40.0 g of sodium hydroxide with 1000.8 g of water for injection.

A 1M hydrochloric acid solution was prepared by combining hydrochloric acid with 1000 g of water for injection.

The buffers were prepared by dissolving the pre-weighed ingredients as described above in 70-90% required weight of the WFI, preferably in the order of disodium phosphate dodecahydrate, sodium dihydrogen phosphate monohydrate, followed by polysorbate 80. Following addition of all of the buffer constituents, the pH of the solution was adjusted with 1 M sodium hydroxide solution or 1M hydrochloric acid solution which was prepared as described above. After the addition of the pH adjustment reagent(s), water was added to final weight. The buffer solution was then filtered through a sterilized filter (PVDF or PES, with pore size ranging from 0.10 to 0.45 μm) into a sterilized receptacle.

For the preparation of the liquid formulations purified SM03 was buffer-exchanged against the Diafiltration Buffer prepared as described above by tangential flow ultrafiltration/diafiltration, nominal flow ultrafiltration/dilution or techniques alike. The molecular weight cutoff rate oof the ultrafiltration membrane or cassette is 10 to 100 kDa. When required, the buffer-exchanged SM03 is concentrated by ultrafiltration to an antibody concentration of approx. 25 mg/ml. After completion of the buffer exchange operation, the surfactant polysorbate 80 was added as Formulation Adjustment Buffer as described above to the antibody solution. Finally the concentrations of antibodies and polysorbate were adjusted to the final SM03 concentration of about 10 mg/ml, 25 mg/ml or 50 mg/ml as specified in the particular formulations further below, and polysorbate concentration of about 0.2 mg/ml, 0.4 mg/ml or 0.8 mg/ml.

All formulations were sterile-filtered through 0.22 μm low protein binding filters and aseptically filled into sterile 30 ml glass vials closed with rubber stoppers and AluCrimp caps. The fill volume was approx. 30 ml. These formulations were stored at different conditions (5±3° C. and 25±2° C.) for different intervals of time and stressed by freeze-thaw stress methods. The samples were analyzed before and after applying the stress tests by the following analytical methods:
1) UV spectrophotometry;
2) Size Exclusion Chromatography (SEC-HPLC);
3) Cation Ion exchange chromatography (CEX-HPLC);
4) visible particles; and
5) antibody activity.

UV spectroscopy, used for determination of protein content, was performed on an UV spectrophotometer at ultraviolet wavelength range from 190 nm to 400 nm. Formulation samples were diluted 20-fold, 30-fold and 40-fold with the corresponding formulation buffer or water. UV spectrophotometer was blanked correspondingly with formulation buffer or water. Absorbance of the diluted samples were measured at 280 nm and 320 nm. The protein concentration was calculated according to below equation.

$$Protein concentration ( mg / ml ) = \frac{A 280 - A 320 \times dilution factor}{ε ( {cm}^{2} / mg ) \times d ( cm )}$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient ε, where the coefficient ranges from 1.3 to 1.5.

Size Exclusion Chromatography (SEC-HPLC) was used to detect antibody monomer (molecular weight approximately 140 kDa to 160 kDa), soluble high molecular weight species (aggregates with molecular weight higher than 200 kDa) and low molecular weight hydrolysis products (LMW, with molecular weight lower than 100 kDa) in the formulations. The method was performed on a Waters Alliance 2695 HPLC instrument with a Waters 2998 PDA Detector and resolved with Waters Protein-Pak 300SW column. Intact monomer, aggregates and hydrolysis products were separated by an isocratic elution profile, using 100 mM sodium acetate, 300 mM sodium chloride, pH 6.5 as mobile phase, and were detected at a wavelength of 280 nm.

Cation Ion Exchange Chromatography (CEX-HPLC) was performed to detect chemical degradation products altering the net charge of SM03 in the formulations. The method used a suitable HPLC instrument equipped with a PDA detector (detection wavelength 280 nm) and a Dionex MabPac™ SCX-10 LC column. 20 mM Bis-Tris buffer pH 6.5 in H₂O and 20 mM Bis-Tris buffer pH 7.5 and 190 mM NaCl were used as mobile phases A and B, respectively, with a flow rate of 0.5 ml/min.

Samples were analyzed for visible particles by using a Beckman HIAC 9703+ particle counter instrument.

### Preparation of 50L of Formulation

The filtered buffer solution was then added to the pooled antibody drug substance prepared as described above. The drug substance, if stored frozen, was thawed in a water bath prior to the preparation of the pharmaceutical formulation or directly being used while stored at 5±3° C. Drug substance with protein concentrate ranging from 10 to 100 mg/mL can be used. The drug substance solution was diluted with Formulation Buffer to target concentration at approximately 10 mg/ml. The buffer was added while stirring, until the final weight of the formulation solution was reached.

The formulation, with protein concentration adjusted to required range, was then sterilized by filtration as described above, except the formulation was filtered through two sterile 0.22 μm membrane filters. Following sterilization, the formulation was packaged for use in glass vial as described above.

The skilled artisan will also appreciate that the weight quantities and/or weight-to-volume ratios recited herein, can be converted to moles and/or molarities using the art-recognized molecular weights of the recited ingredients. Weight quantities exemplified herein (e.g., g or kg) are for the volumes (e.g., of buffer or pharmaceutical formulation) recited. The skilled artisan will appreciate that the weight quantities can be proportionally adjusted when different formulation volumes are desired. For example, 40 L, 25 L, 12.5 L, 5 L or 2.5 L formulations would include 80%, 50%, 25%, 10%, or 5%, respectively, of the exemplified weight quantities.

TABLE 2
Stability data of liquid anti-CD22 drug product formulations according to the present invention

| | | | | | | | CEX-HPLC | | | | | |
|---|---|---|---|---|---|---|---|---|---|---|---|---|
| | | | | SEC-HPLC | | | | | | Acidic | Binding | Biological |
| Storage | Storage | | Concentration | HMW | Monomer | LMW | 0K | 1K | 2K | fraction | activity | activity |
| temp. | time | | mg/ml | % | % | % | % | % | % | % | U/mg | U/mg |
| — | Initial | | 10.1 | 2.3 | 97.5 | 0.2 | 69.83 | 16.86 | 5.75 | 7.19 | 8949 | 11030 |
| 5° C. | 3 | months | 10.2 | 2.4 | 97.4 | 0.2 | 67.05 | 16.97 | 5.7 | 8.84 | 8559 | 8443 |
| | 6 | months | 10.1 | 2.3 | 97.5 | 0.2 | 67.44 | 16.82 | 5.68 | 8.76 | 9641 | 8882 |
| | 12 | months | 10.0 | 2.2 | 97.6 | 0.2 | 66.08 | 16.61 | 5.64 | 10.04 | 7279 | 10189 |
| | 18 | months | 10.0 | 2.4 | 97.4 | 0.2 | 69.32 | 15.35 | 5.37 | 10.7 | 10444 | 7149 |
| | 24 | months | 10.1 | 2.7 | 97.0 | 0.3 | 68.42 | 14.98 | 4.8 | 11.8 | 8010 | 10257 |
| | 36 | months | 10.1 | 2.9 | 96.7 | 0.4 | 63.96 | 15.62 | 5.15 | 13.57 | 9014 | 8999 |
| 25° C. | 1 | month | 10.0 | 2.5 | 97.3 | 0.2 | 67.15 | 16.93 | 5.73 | 8.1 | 9781 | 9922 |
| | 3 | months | 10.0 | 3.5 | 96.3 | 0.2 | 64.11 | 15.82 | 5.23 | 10.21 | 8385 | 11322 |
| | 6 | months | 10.1 | 4.5 | 95.1 | 0.4 | 63.23 | 15.73 | 5.08 | 13.62 | 9603 | 8572 |

### Example 2—Freeze/Thaw Studies

Freeze thaw behavior of the anti-CD22 antibody drug formulation at a protein concentration of 10 mg/mL was evaluated by cycling drug formulation 3 times from the frozen state to the liquid state. Table 3 shows the results of an experiment evaluating the effect of three freeze-thaw cycles starting from −80° C. or −30° C., respectively. Table 3 shows that the D2E7 antibody drug substance can be thawed/frozen at least 3 times without any detrimental effect on either chemical (cation exchange HPLC, size exclusion HPLC, colour, pH), or biological activity (in vitro TNF neutralization assay).

TABLE 3
Chemical Properties of Anti-CD22 drug formulations
after multiple cycles of freezing & thawing

| | No F/T | F/T 1 cycle | F/T 2 cycles | F/T 3 cycles |
|---|---|---|---|---|
| Concentration | 10.162 | 10.100 | 10.172 | 10.224 |
| Monomer | 97.5 | 96.8 | 97.4 | 96.0 |
| Aggregate | 2.1 | 2.9 | 2.3 | 3.6 |
| Activity (%) | 100 | 92.9 | 98.8 | 88.6 |

### Example 3—Treatment with Anti-CD22 Formulation Via Intravenous Administration

In this open label study, eight patients with active RA were treated with two injections of 600 mg per dose (roughly equivalent to 360 mg/m 2) of SM03 on day 0 and day 15, in addition to their on-going treatment with (methotrexate) MTX. Patients participating in this study had a mean duration of disease of 5.8 years with a disease activity score (DAS28) of 5.78, reflecting considerable disease activity.

Four of the eight patients treated with SM03 achieved a DAS28-based EULAR response and ACR20 over twelve weeks, with the earliest response onset on the fourth week; one of them achieved ACR70 (Table 4 below).

TABLE 4
Percentage of ACR responders following administration
of one kit of SM03 (Two vials, 600 mg total)
during an Open - label Phase I study

| ACR | Week 4 | Week 8 | Week 12 |
|---|---|---|---|
| ACR20 | 2 (25%) | 3 (37.5%) | 4 (50%) |
| ACR50 | 0 | 0 | 1 (12.5%) |
| ACR70 | 0 | 0 | 1 (12.5%) |

Two of the responders began to observe ACR improvement on day 28, and the remaining responders did not achieve ACR responses until Day 56 and Day 84, respectively. Unlike other TNFα-targeting antibodies, which could demonstrate ACR responses in as early as the first week after antibody administration, indicating that anti-CD22 responses against RA might be slow-acting.

In agreement with the ACR scores, DAS28 scores for enrolled patients trended lower with time, albeit slowly, with the DAS28 score achieving its lowest value (down by a value of approximately 0.6) on DAY 84 post SM03 administration. Four of the eight patients achieved DAS EULAR responses on day 84, of which one was considered good EULAR response. Regarding the average baseline value for high activity RA patients of over 5.1, the change in DAS28 score observed did not reflect responses to the SM03 treatment.

If a reduction of DAS28 score of individual patients by over 0.6-1.2 was being considered as reflecting “moderate responses”, two patients treated with SM03 could be considered as having achieved moderate responses. Although two other patients achieved DAS28 reduction by over 1.2, one on Day 28 and the other on Day 56, the score bounced back upon subsequent follow-up visits. Regardless, the results indicated that i.v. dosing of 600 mg of SM03 were effective for treating RA with a good safety and tolerability profile.

### EXAMPLE 4—Total Body Dose of an Intravenously Administered Anti-CD22 Antibody Formulation

Bi-weekly, Intravenous Administration of SM03 in two cycles The clinical effects, safety, immunogenicity, and tolerance of RA patients with insufficient response to MTX following biweekly intravenous injections of placebo or SM03 at two cumulative dose levels in two cycles, each with two or three injections in conjunction with continued MTX treatment was investigated.

A placebo-controlled, double-blind, randomized, multicenter study in patients with RA, who had insufficient response or tolerability to MTX was performed. During the course of the trial, patients were continued on a stable dose of MTX with dose ranges specified in the inclusion criteria described below.

The study consisted of two portions: 1) a “wash-out period” of four weeks prior to the administration of the first dose medication, during which time DMARDs (except for MTX) were withdrawn; and 2) a “placebo controlled period” during which time patients were randomized to one of three cohorts of fifty-two patients to receive placebo, 2×600 mg SM03 in two cycles, or 3×600 mg SM03, given every other week, in two cycles with the first cycle starting at week 0 and the second cycle at week 12.

Patients were serially examined in weeks zero (before SM03 administration), two, four, eight, twelve, sixteen and twenty-four of the study with the joint examinations being performed by a blinded physician (investigator).

This study enrolled one hundred and fifty-six patients with RA. The study population was representative of the moderate to severe RA population in Northern, West Southern, and Eastern part of China: approximately 86% female, and predominantly over the age of forty-four. The population was selected using predetermined inclusion and exclusion criteria, known to those of skill in the art e.g., a patient must have received a diagnosis of RA as defined by the 1987-revised American College of Rheumatology (ACR) criteria (see, e.g., Arnett F. C., et al. (1988), “The American Rheumatism Association 1987 revised criteria for the classification of rheumatoid arthritis”, Arthritis Rheum . 31: 314-324).

### Results

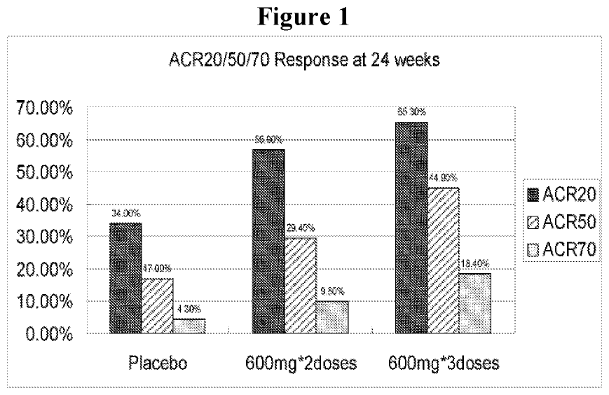

FIG. 1 indicate that intravenous, biweekly SM03 treatment in two cycles, either with two biweekly administration or three biweekly administration, when combined with methotrexate, was significantly better than placebo in reducing the signs and symptoms of RA at twenty-four weeks. Interestingly, all dosing regimens of SM03 were significantly more effective than placebo given following the same dosing schedules of the treatment groups, when assessed with ACR20 responses. Specifically, at week 24, approximately 57% of patients treated with 2×600 mg of SM03, and 65% of patients treated with 3×600 mg of SM03 achieved ACR20, whereas only 34% of placebo achieved ACR20; furthermore, roughly 30% and 45% of patients treated with 2×600 mg and 3×600 mg SM03, respectively, achieved ACR50 (versus 17% of patients in the placebo group with ACR50 response); and roughly 10% and 18.4% of patients treated with 2×600 mg and 3×600 mg SM03, respectively, achieved ACR70 (versus 4% of patients in the placebo group with ACR70 response).

All publications, patent applications, patents and other references mentioned herein are incorporated by reference in their entirety.

While the invention has been described in detail and with reference to specific embodiments thereof, it will be apparent to one skilled in the art that various changes and modifications can be made therein without departing from the spirit and scope of the invention.

1. A stable liquid pharmaceutical formulation of a therapeutically active anti-CD22 antibody, said formulation comprising:
(a) an IgG1 anti-human CD22 antibody, or a protein containing an antigen-binding portion thereof, at a concentration of 5 to 25 mg/ml;
(b) 1 to 50 mM of a buffering agent providing a pH of 5.8 to 8.0, and
(c) 0.01 to 0.08% of a nonionic surfactant,
wherein said antibody is Suciraslimab (SM03).

2. The formulation of claim 1, wherein the concentration of said IgG1 anti-human CD22 antibody or protein containing an antigen-binding portion thereof is 10 mg/ml.

3. The formulation of claim 1, wherein the formulation has an osmotic pressure that ranges from 50 to 100 mOsmol/kg.

4. The formulation of claim 1, wherein said nonionic surfactant is at a concentration of 0.01 to 0.05%.

5. The formulation of claim 1, wherein the surfactant is a polysorbate.

6. The formulation of claim 5, wherein the polysorbate is polysorbate 20.

7. The formulation of claim 5, wherein the polysorbate is polysorbate 80.

8. The formulation of claim 7, wherein the polysorbate 80 concentration ranges from 0.01 to 0.05%.

9. The formulation of claim 8, wherein the polysorbate 80 concentration is about 0.04%.

10. The formulation of claim 1, wherein the pH ranges from 6.9 to 7.5.

11. The formulation of claim 1, wherein said formulation is suitable for injection.

12. The formulation of claim 1, wherein said formulation is suitable for intravenous infusion.

13. The formulation according to claim 1, wherein said buffering agent is a phosphate buffer.

14. The formulation of claim 1, wherein said anti-CD22 antibody comprises:
a. a variable light chain region comprised of the amino acid sequence of SEQ ID NO: 2, an amino acid sequence of SEQ ID NO: 3, and the amino acid sequence of SEQ ID NO: 4; and
b. a variable heavy chain region comprised of the amino acid sequence of SEQ ID NO: 6, the amino acid sequence of SEQ ID NO: 7, and the amino acid sequence of SEQ ID NO: 8.

15. The formulation of claim 1, wherein said anti-CD22 antibody comprises:
a. a variable light chain region comprising the amino acid sequence of SEQ ID NO: 1; and
b. a variable heavy chain region comprising the amino acid sequence of SEQ ID NO: 5.

16. The formulation according to claim 1, wherein said formulation is stable upon multiple cycles of freezing and thawing.

17. The formulation according to claim 1, wherein said formulation is in a form suitable for the treatment of a disease or disorder amenable to treatment with an anti-CD22 antibody.

18. The injection according to claim 1, wherein said formulation is co-administered concomitantly with an isotonic agent in a preloaded syringe that comprises a therapeutically effective amount of SM03.

19. A method of treating an autoimmune disorder or disease in a subject in need thereof, said method comprising the step of administering the formulation according to claim 1 to said subject in an amount effective to bind and/or inhibit B-lymphocytes.

20. The method according to claim 19, wherein said autoimmune disorder or disease is selected from the group consisting of rheumatoid arthritis (RA), rheumatoid spondylitis, systemic lupus erythematosus (SLE), multiple sclerosis (MS), autoimmune diabetes, Sjogren's syndrome, psoriasis, inflammatory bowel disease, Graves' disease, diabetes mellitus type 1, celiac diseases, osteoarthritis, gouty arthritis, allergy, autoimmune uveitis, nephrotic syndrome, pemphigus vulgaris, Alzheimer's Disease, Behcet diseases (BD), and B cell malignancy.

21. A method of treating autoimmune disorder or disease in a subject in need thereof, said method comprising the step of administering the formulation according to claim 14 to said subject in an amount effective to bind and/or inhibit B-lymphocytes.

22. The method according to claim 21, wherein said autoimmune disorder or disease is associated with disrupted immune self-tolerance.

23. The method according to claim 22, wherein said autoimmune disorder or disease is selected from the group consisting of rheumatoid arthritis, rheumatoid spondylitis, osteoarthritis, gouty arthritis, allergy, multiple sclerosis, autoimmune diabetes, autoimmune uveitis and nephrotic syndrome.

24. A kit comprising one or more vials containing the formulation according to claim 1 and instructions for intravenous administration of the formulation to a patient in need thereof.


SEQUENCE LISTING (10 sequences)

SEQ ID NO: 1 - protein, 108 aa, synthetic construct
DIQLTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKR

SEQ ID NO: 2 - protein, 11 aa, synthetic construct
RASQDISNYLN

SEQ ID NO: 3 - protein, 7 aa, synthetic construct
YTSILHS

SEQ ID NO: 4 - protein, 9 aa, synthetic construct
QQGNTLPWT

SEQ ID NO: 5 - protein, 123 aa, synthetic construct
QVQLQESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSS

SEQ ID NO: 6 - protein, 5 aa, synthetic construct
IYDMS

SEQ ID NO: 7 - protein, 17 aa, synthetic construct
YISSGGGTTYYPDTVKG

SEQ ID NO: 8 - protein, 14 aa, synthetic construct
HSGYGSSYGVLFAY

SEQ ID NO: 9 - protein, 108 aa, synthetic construct
DIQLTQSPSSLSASVGDRVTISCRASQDISNYLNWYQQKPGKAPKLLIYYTSILHSGVPSRFSGSGSGTEFTLTISSLQPEDFATYFCQQGNTLPWTFGGGTKVEIKR

SEQ ID NO: 10 - protein, 123 aa, synthetic construct
EVQLVESGGGLVQPGGSLRLSCAASGFSFSIYDMSWVRQAPGKGLEWVAYISSGGGTTYYPDTVKGRFTISRDNAKNSLYLQMNSLRVEDTALYYCARHSGYGSSYGVLFAYWGQGTLVTVSS